(19) United States
(12) Patent Application Publication
(10) Pub. No.: US 2015/0155106 A1
(43) Pub. Date: Jun. 4, 2015

(54) AGENT FOR FORMING ELECTRODE PROTECTION FILM

(71) Applicant: SANYO CHEMICAL INDUSTRIES, LTD., KYOTO (JP)

(72) Inventors: Junko Takada, Kyoto (JP); Fumiyuki Tanabe, Kyoto (JP)

(21) Appl. No.: 14/411,911

(22) PCT Filed: Jun. 24, 2013
(86) PCT No.: PCT/JP2013/003929

§ 371 Date: Dec. 29, 2014

(30) Foreign Application Priority Data
Jul. 5, 2012 (JP) 2012-150976
Jul. 10, 2012 (JP) 2012-154603
Nov. 16, 2012 (JP) 2012-252481
Mar. 27, 2013 (JP) 2013-065590

(51) Int. Cl. H01G 11/00; C25D 9/02; H01M 10/42; C08F 116/12; C07C 271/24; C07D 251/34; C08F 116/14; H01M 10/0567; C07C 271/12

(52) U.S. Cl. CPC H01G 11/00; H01M 10/0567; C25D 9/02; H01M 10/4235; C07C 271/12; C07C 271/24; C07D 251/34; C08F 116/14; C08F 116/12; H01M 2300/0025; H01M 2220/30

(57) ABSTRACT

Described is an electrode or electrolyte solution for an electrochemical device that can be used in a wider temperature range with good long-term stability. An electrode protection film forming agent contains a compound having a urethane bond and a polymerizable unsaturated bond and preferably represented by general formula (1): A(-NHCO₂—X)ₙ. A is i) an n-valent C₂₋₄₂ hydrocarbon group, ii) a trivalent residue obtained by removing three isocyanate groups from a trimer of a C₂₋₄₂ diisocyanate (B), or iii) a divalent residue obtained by removing two isocyanate groups from a urethane prepolymer having isocyanate groups at both ends and being a reaction product of C₂₋₄₂ diisocyanate (B) and C₂₋₂₀ diol (N). X is a monovalent C₃₋₄₂ organic group having a polymerizable unsaturated bond (b), n is an integer of 1 to 6, and when n is 2 or more, multiple X may be the same or different from each other.

### FIELD OF THE INVENTION

The invention relates to an additive adapted for use in an electrode and an electrolyte solution for electrochemical devices, and also to an electrode and an electrolyte solution that use the additive. More specifically, the invention relates to an electrode protection film forming agent useful in lithium secondary batteries, lithium-ion capacitors or electrical double-layer capacitors, and also to an electrode and an electrolyte solution that use the same.

### DESCRIPTION OF THE RELATED ART

With features of high voltage and high energy density, nonaqueous electrolyte secondary batteries such as lithium secondary batteries, etc. are widely used in the field of portable digital devices or the like, and demand therefor is rapidly increasing. Currently, the nonaqueous electrolyte secondary batteries have been established as standard batteries for mobile digital devices including mobile phones, notebook computers, etc. Understandably, as portable devices or the like are improved in performance and function, the nonaqueous electrolyte secondary batteries as power sources of the portable devices or the like are also required to show further improved performance (e.g., higher capacity and higher energy density). To meet such requirement, various methods, e.g., high densification by increasing the filling ratio of the electrode, increasing the depth of use of an existing active material (especially for the negative electrode) and developing new high-capacity active materials, etc., have been developed. Practically, the nonaqueous electrolyte secondary batteries actually achieve high capacity based on these methods.

In addition, for even higher capacity of nonaqueous electrolyte secondary batteries, improvement in utilization ratio of the positive electrode active material or development of high-voltage materials are required. Among these, particularly the increase of the depth of use of the positive electrode active material by increasing a charging voltage attracts attention. For example, a cobalt composite oxide (LiCoO₂) as an active material of a nonaqueous electrolyte secondary battery having an operating voltage level of 4.2 V has a charging capacity of about 155 mAh/g when charged to 4.3 V based on current Li standard, while having a charging capacity of about 190 mAh/g or higher when charged to 4.50 V. In this manner, because of the increase of the charging voltage, the utilization ratio of the positive electrode active material is increased.

However, since a nonaqueous electrolyte solution is easily decomposed due to the increase of the charging voltage, a problem has been encountered in that charge/discharge cycle characteristic deteriorates or the battery may swell as gas such as carbon dioxide is generated during high-temperature storage.

An electrical double-layer capacitor has lower voltage and energy density as compared to a lithium secondary battery, but on the other hand, is capable of being charged and discharged in a shorter time than a lithium secondary battery. Therefore, widespread utilization of electrical double-layer capacitors as a backup power supply or a power supply of a hybrid electric vehicle is expected.

However, in new application fields such as hybrid electric vehicles or the like that are used at a large current under severe conditions, there is a demand for an electrochemical device that can be used in a wider temperature range with good long-term stability.

In order to improve the performances of the electrochemical devices including lithium secondary batteries, lithium-ion capacitors and electrical double-layer capacitors, various techniques for improving the electrodes or the electrolyte solutions constituting these electrochemical devices have been proposed.

With respect to the nonaqueous electrolyte secondary battery, in Patent Document 1, an aromatic sulfide such as methyl phenyl sulfide or diphenyl sulfide, etc. is added, and is preferentially oxidized over an electrolyte solution on the surface of the positive electrode. By repeating a reaction of diffusing the product of the oxidation to the negative electrode and reducing the same into the original sulfide, oxidative decomposition of solvent is suppressed. It is disclosed that due to this reaction, the storage characteristic, charge/discharge cycle characteristic and so on are improved.

Patent Document 2 discloses that by adding a sulfide compound having an aryl group or a heterocyclic group as a substituent that preferentially reacts with the strongly oxidative chemical species such as active oxygen, etc. generated on the surface of the positive electrode so as to suppress oxidative decomposition of the solvent, reduction in discharging capacity caused by repetition of charge and discharge is suppressed. Furthermore, it is also disclosed that an oxidized portion is attached to the positive electrode and reduced to its original state during discharge, and that another portion is diffused to the negative electrode.

With respect to an electrical double-layer capacitor, Patent Document 3 discloses that by adding to the electrolyte solution a glycol diether that is absorbed by the surface of the electrode so as to suppress decomposition of the electrolyte solution, the capacity reduction is suppressed and the durability is improved.

Patent Document 4 discloses that by adding an imidazolium salt having a vinyl group to an electrolyte solution, the capacity reduction or resistance increase after a long-term use is reduced, and the cycle characteristic and long-term durability are improved.

### PRIOR-ART DOCUMENTS

### Patent Documents

Patent Document 1: JPH07-320779 A

Patent Document 2: JPH10-64591 A

Patent Document 3: JP2011-204918 A

Patent Document 4: JP2011-151237 A

### SUMMARY OF THE INVENTION

### Problems to be Solved by the Invention

However, when a sulfide compound like that of Patent Documents 1 or 2 is used in a lithium secondary battery, there are problems including that the sulfide compound itself may be decomposed into a radical, and that the cycle characteristic may deteriorate due to the reaction with the electrolyte solution or the electrode.

In addition, even if a compound like that of Patent Documents 3 or 4 is used in an electrical double-layer capacitor, it is not satisfactory in terms of the effect of improving the long-term durability.

The invention aims to provide an electrode or electrolyte solution for electrochemical devices that can be used in a wider temperature range with good long-term stability.

### Means for Solving the Problems

As a result of earnest studies to achieve the above-mentioned object, the Inventors have attained the invention. Specifically, the invention includes: an electrode protection film forming agent (D) comprising a compound (C) having a urethane bond (a) and a polymerizable unsaturated bond (b), an electrode containing the electrode protection film forming agent (D), an electrolyte solution containing the electrode protection film forming agent (D), a lithium secondary battery including the above electrode and/or the above electrolyte solution, a lithium-ion capacitor including the above electrode and/or the above electrolyte solution, an electrical double-layer capacitor including the above electrode and/or the above electrolyte solution, and a method for fabricating an electrode protection film that includes a step of applying a voltage after the electrode protection film forming agent (D) is contained in the electrode and/or the electrolyte solution.

### Effects of the Invention

By using the electrode or electrolyte solution that contains the electrode protection film forming agent of the invention, an electrochemical device that can be used in a wider temperature range with good long-term stability can be obtained. More specifically, with respect to a lithium secondary battery and a lithium-ion capacitor, charge/discharge cycle performance and high-temperature storage characteristic can be improved. In addition, with respect to an electrical double-layer capacitor, the long-term durability can be improved.

### DESCRIPTION OF THE EMBODIMENTS

<Electrode Protection Film Forming Agent (D)>

The electrode protection film forming agent (D) of the invention is contained in the negative electrode, the positive electrode or both electrodes of a lithium secondary battery, a lithium-ion capacitor or an electrical double-layer capacitor, and then forms a polymer film on the surface of the electrode active material when a voltage is applied. Due to the effect of the polymer film, the charge/discharge cycle performance and high-temperature storage characteristic of the lithium secondary battery or the lithium-ion capacitor can be improved, or the long-term durability of the electrical double-layer capacitor can be improved.

In addition, (D) is contained in the electrolyte solution of a lithium secondary battery, a lithium-ion capacitor or an electrical double-layer capacitor, and then forms a polymer film on the surface of the electrode active material when a voltage is applied. Due to the effect of the polymer film, the charge/discharge cycle performance and high-temperature storage characteristic of the lithium secondary battery or the lithium-ion capacitor can be improved, or the long-term durability of the electrical double-layer capacitor can be improved.

The electrode protection film forming agent (D) of the invention is characterized by containing a compound (C) with a urethane bond (a) and a polymerizable unsaturated bond (b).

The compound (C) is preferably represented by the following general formula (1).

A(-NHCO₂—X)ₙ (1)

In general formula (1), A is i) an n-valent hydrocarbon group (A1) having 2 to 42 carbon atoms, ii) a trivalent residue (A2) obtained by removing three isocyanate groups from a trimer of a diisocyanate (B) having 2 to 42 carbon atoms, or iii) a divalent residue (A3) obtained by removing two isocyanate groups from a urethane prepolymer that has isocyanate groups at both ends and is a reaction product of a diisocyanate (B) having 2 to 42 carbon atoms and a diol (N) having 2 to 20 carbon atoms;

X is a monovalent organic group having 3 to 42 carbon atoms and having a polymerizable unsaturated bond (b), n is an integer of 1 to 6, and when n is 2 or more, a plurality of X may be the same or different from each other.

(A1) is exemplified by the following groups:

a monovalent aliphatic hydrocarbon group, specific examples thereof including n-butyl group, etc.;
a divalent aliphatic hydrocarbon group, preferably a polymethylene group having 2 to 10 carbon atoms, specific examples thereof including methylene, ethylene, tetramethylene, hexamethylene, octamethylene, decamethylene, 1-methyltetramethyl ene, and 2-methyltetramethylene, etc.;

a divalent alicyclic hydrocarbon group, preferably an alicyclic hydrocarbon group having 5 to 13 carbon atoms, specific examples thereof including 1,5,5-trimethyl-cyclohexane-1,3-diyl, methylene dicyclohexyl-4,4′-diyl, cyclohexane-1,4-diyl, and 1,4-dimethylene-cyclohexane (residue obtained by removing two hydroxyl groups from 1,4-cyclohexanedimethanol), etc.; and

a divalent aromatic hydrocarbon group, preferably an aromatic hydrocarbon group having 6 to 12 carbon atoms or an aliphatic-aromatic hydrocarbon group having 6 to 42 carbon atoms, specific examples thereof including toluene-2,4-diyl, toluene-2,6-diyl, methylenediphenyl-4,4′-diyl, xylylene, tetramethylxylylene, phenylene, and 1,5-naphthalene, etc.

(A2) is exemplified by a trivalent residue obtained by removing three isocyanate groups from a trimer of ethylenediisocyanate, a trimer of hexamethylenediisocyanate or a trimer of isophorone diisocyanate.

The diisocyanate (B) having 2 to 42 carbon atoms is exemplified by:

an aliphatic hydrocarbon diisocyanate (B1), such as ethylenediisocyanate, or hexamethylenediisocyanate, etc.;

an alicyclic hydrocarbon diisocyanate (B2), such as dicyclohexylmethane4,4′-diisocyanate, or isophorone didiisocyanate, etc.;

an aromatic hydrocarbon diisocyanate (B3), such as diphenylmethane diisocyanate, or toluene diisocyanate, etc.; and

an aliphatic-aromatic hydrocarbon diisocyanate (B4), such as xylylene diisocyanate, or α,α,α′,α′-tetramethylxylylene diisocyanate, etc.

The diol (N) having 2 to 20 carbon atoms is exemplified by 1,4-butanediol, 1,6-hexanediol, 3-methyl-1,5-pentanediol, 1,4-cyclohexanedimethanol, and 1,4-cyclohexanediethanol, etc.

When the urethane prepolymer being a reaction product of the diisocyanate (B) and the diol (N) and having isocyanate groups at both ends is represented by

B—(N—B)ₘ—N—B,

it is preferably a prepolymer with m being 0 to 10.

X is a monovalent organic group having 3 to 42 carbon atoms, preferably 5 to 20 carbon atoms, and having a polymerizable unsaturated bond (b). The polymerizable unsaturated bond (b) is exemplified by a C—C double bond, a C—C triple bond, a C—N double bond, a C—N triple bond, etc.;

n is an integer of 1 to 6, preferably 1 to 3; when n is 2 or more, a plurality of X may be the same or different from each other.

X is preferably the following (X1) to (X3):

a monovalent aliphatic hydrocarbon group (X1) having 3 to 42 carbon atoms, containing one to four C—C double bonds and may containing a ring other than an aromatic ring;

a monovalent hydrocarbon group (X2) having 8 to 42 carbon atoms, containing one to four C—C double bonds and having an aromatic ring; and

a monovalent organic group (X3) having 3 to 42 carbon atoms, containing one to four C—C double bonds of which at least one is in a bond represented by the following formula (2), or in an acryloyloxyalkyl group or a methacryloyloxyalkyl group.

C═C—O (2)

Among them, (X1) is more preferred, and among groups (X1), one having a structure represented by the following formula (3) is preferred.

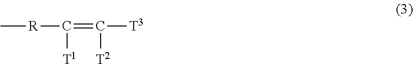

In formula (3), T¹ to T³ are a hydrogen atom or an alkyl group having 1 to 3 carbon atoms, at least two of T¹ to T³ are alkyl groups having 1 to 3 carbon atoms and may form a ring with each other, and R is a divalent hydrocarbon group having 1 to 12 carbon atoms.

Specific examples of (X1) are 3-methyl-2-butenyl, a residue obtained by removing the hydroxyl group from linalool, a residue obtained by removing the hydroxyl group from citronellol, a residue obtained by removing the hydroxyl group from geraniol, and a residue obtained by removing the hydroxyl group from retinol, etc.

(X2) is preferably one obtained by conjugating a C—C double bond with an aromatic ring, being exemplified by 3-phenyl-2-propenyl, (E)-2-methyl-3-phenyl-2-propenyl, and (4-ethenylphenyl)methyl, etc.

(X3) is exemplified by [4-(1-propenoxymethyl)cyclohexyl]methyl, [4-(1-butenoxymethyl)cyclohexyl]methyl, 4-(1-propenoxyl)butyl, 6-(1-propenoxyl)hexyl, 6-(2-methyl-1-propenoxy)hexyl, acryloyloxyethyl, and methacryloyloxyethyl, etc.

The compound (C) can be synthesized by reacting an isocyanate compound (G) having the structure A with an active-hydrogen compound (H) having the polymerizable unsaturated bond (b), in the presence or absence of a urethane-forming catalyst.

The isocyanate compound (G) is exemplified by the following (G1) to (G3):

a monoisocyanate compound (G1) having a monovalent hydrocarbon group having 2 to 42 carbon atoms, such as butyl isocyanate, etc.;

a diisocyanate compound (G2) having a divalent hydrocarbon group having 2 to 42 carbon atoms, such as the same compound as the above-mentioned diisocyanate (B) having 2 to 42 carbon atoms, and a urethane prepolymer being a reaction product of the above-mentioned diisocyanate (B) and the above-mentioned diol (N) and having isocyanate groups at both ends, for example, a urethane prepolymer being a reaction product of hexamethylenediisocyanate and 1,6-hexanediol and having isocyanate groups at both ends, etc.; and

a triisocyanate compound (G3) having 12 to 60 carbon atoms, such as a trimer of ethylenediisocyanate, a trimer of hexamethylenediisocyanate, and a trimer of isophoronediisocyanate, etc.

The active hydrogen compound (H) is an active hydrogen compound represented by X—OH, X—NH₂, X—SH, etc. Among them, in view of the reactivity with isocyanate, X—OH is preferred. Specific examples of the active-hydrogen compound (H) having the residue (X1) include 3-methyl-2-buten-1-ol, linalool, citronellol, geraniol, and retinol, etc.

Specific examples of the active hydrogen compound (H) having the residue (X2) include cinnamyl alcohol, (E)-2-methyl-3-phenyl-2-propen-1-ol, and (4-ethenylphenyl)methanol, etc.

Specific examples of the active hydrogen compound (H) having the residue (X3) include 1-hydroxymethyl-4-(1-propenoxymethyl)cyclohexane, 1-hydroxymethyl-4-(1-butenoxymethyl)cyclohexane, 4-(1-propenoxyl)butan-1-ol, 6-(1-propenoxyl)hexan-1-ol, 6-(2-methyl-1-propenoxy)hexan-1-ol, 2-hydroxyethyl acrylate, and 2-hydroxyethyl methacrylate, etc.

The concentration of the urethane bond (a) in the compound (C) is preferably 0.2 to 7.5 mmol/g, and more preferably 2.0 to 5.0 mmol/g. The concentration of (a) is preferably not less than 0.2 mmol/g in view of the cycle characteristic, and preferably not more than 7.5 mmol/g in view of the solubility in the electrolyte solution.

The concentration of the polymerizable unsaturated bond (b) in the compound (C) is preferably 0.2 to 15.0 mmol/g, and more preferably 2.0 to 8.0 mmol/g. The concentration of (b) is preferably not less than 0.2 mmol/g in view of the cycle characteristic, and preferably not more than 15.0 mmol/g in view of the electrode interface resistance.

The number average molecular weight of the compound (C) is preferably not more than 5000, more preferably not more than 3500, in view of the solubility in a later-described dispersion solvent. The number average molecular weight of (C) is measured by gel permeation chromatography (hereinafter referred to as GPC). The measurement conditions include, e.g., using tetrahydrofuran (THF) as a solvent at 40° C. The molecular weight can be measured by means of a mass spectrometer or calculated from the structural formula.

The electrode protection film forming agent (D) may contain components other than the compound (C), but preferably contains no component other than the compound (C). The components other than the compound (C) are exemplified by a Lewis base (I), and a negative electrode protection film forming agent (J), etc. The Lewis base (I) is exemplified by a triazole derivative (1,2,3-benzotriazole, 5-methyl-1,2,3-benzotriazole, 5,6-dimethyl-1,2,3-benzotriazole, 1,2,4-triazole, 3-amino-1,2,4-triazole, 3,5-diamino-1,2,4-triazole, 3-amino-5-methyl-1,2,4-triazole, 3-amino-5-ethyl-1,2,4-triazole, 3-amino-5-propyl-1,2,4-triazole, and 3-amino-5-butyl-1,2,4-triazole, etc.). The negative electrode protection film forming agent (J) is exemplified by vinylene carbonate, fluoroethylene carbonate, chloroethylene carbonate, ethylene sulfite, propylene sulfite, and α-bromo-γ-butyrolactone, etc.

The content of the compound (C) in the electrode protection film forming agent (D) is preferably 10 to 100 wt %, and more preferably 50 to 100 wt %, based on the weight of (D).

<Electrode>

The electrode of the invention contains the electrode protection film forming agent (D) and an active material (Q) before being charged or discharged in use, preferably also contains a binder (K). As a charge/discharge process starts, a part of (D) undergoes a polymerization reaction to form a polymer film on the surface of (Q). At this moment, the electrode of the invention contains an unreacted electrode protection film forming agent (D), and the active material (Q) having an electrode protection film that includes the polymer of (D) formed on its surface, preferably also contains the binder (K). Furthermore, if the charge/discharge process continues, (D) may all become a polymer film.

The positive electrode active material (Q11) for lithium secondary batteries is exemplified by a composite oxide of lithium and a transition metal (e.g., LiCoO₂, LiNiO₂, LiMnO₇ and LiMn₂O₄), a transition metal oxide (e.g., MnO₂ and V₂O₅), a transition metal sulfide (e.g., MoS₂ and TiS₂), and a conductive polymer (e.g., polyaniline, polyvinylidene fluoride, polypyrrole, polythiophene, polyacetylene, poly-p-phenylene, polycarbazole), etc.

The negative electrode active material (Q12) for lithium secondary batteries is exemplified by graphite, amorphous carbon, a polymer compound calcined material (e.g., a carbonized material obtained by calcining a phenolic resin or furan resin, etc.), cokes (e.g., pitch coke, needle coke and petroleum coke), a carbon fiber, a conductive polymer (e.g., polyacetylene and polypyrrole), tin, silicon, and metal alloy (e.g., lithium-tin alloy, lithium-silicon alloy, lithium-aluminum alloy and lithium-aluminum-manganese alloy, etc.), etc.

The positive electrode active material (Q21) for lithium-ion capacitors is exemplified by a carbon material (sawdust activated carbon, coconut shell activated carbon, pitch coke-based activated carbon, phenolic resin-based activated carbon, polyacrylonitrile-based activated carbon, and cellulose-based activated carbon, etc.), a carbon fiber, a metal oxide (ruthenium oxide, manganese oxide, and cobalt oxide, etc.) and a conductive polymer material (polyaniline, polypyrrole, polythiophene, and polyacetylene, etc.).

The negative electrode active material (Q22) for lithium-ion capacitors is obtained by doping lithium in the negative electrode active material (Q12) for lithium secondary batteries.

The positive electrode active material and the negative electrode active material (Q3) for electrical double-layer capacitors adopt the same substance as the positive electrode active material (Q21) for lithium-ion capacitors.

The binder (K) is exemplified by a polymeric compound such as starch, polyvinylidene fluoride, polyvinyl alcohol, carboxymethyl cellulose, polyvinylpyrrolidone, tetrafluoroethylene, polyethylene, and polypropylene, etc.

The electrode of the invention can further contain a conduction aid (L).

The conduction aid (L) is exemplified by graphite (e.g., natural graphite and artificial graphite) (except cases where graphite is used as the active material (Q)), carbon blacks (e.g., carbon black, acetylene black, Ketjen black, channel black, furnace black, lamp black, and thermal black) and metal powder (e.g., aluminum powder and nickel powder), a conductive metal oxide (e.g., zinc oxide and titanium oxide), etc.

Based on the total weight of the electrode protection film forming agent (D), the active material (Q) and the binder (K) in the electrode of the invention, preferred contents of the electrode protection film forming agent (D), the active material (Q), the binder (K) and the conduction aid (L) are respectively described as follows.

In view of charge/discharge cycle characteristic, the content of the electrode protection film forming agent (D) is preferably 0.05 to 5 wt %, and more preferably 0.1 to 2 wt %.

In view of the battery capacity, the content of the active material (Q) is preferably 70 to 98 wt %, and more preferably 90 to 98 wt %.

In view of the battery capacity, the content of the binder (K) is preferably 0.5 to 29 wt %, and more preferably 1 to 10 wt %.

In view of the battery output, the content of the conduction aid (L) is preferably 0 to 29 wt %, and more preferably 1 to 10 wt %.

The electrode of the invention is obtained by the following steps, for example. The electrode protection film forming agent (D), the active material (Q), the binder (K), and the conduction aid (L), if necessary, are dispersed in water or a solvent at a concentration of 20 to 60 wt % to form a slurry. The slurry is coated onto a current collector by means of a coating device such as a bar coater, and then dried to remove the solvent, and, if necessary, pressed by means of a pressing machine.

As the above-mentioned dispersion solvent, a lactam compound, a ketone compound, an amide compound, an amine compound, or a cyclic ether compound, etc. can be used.

Examples thereof include 1-methyl-2-pyrrolidone, methyl ethyl ketone, dimethylformamide, dimethylacetamide, N,N-dimethylaminopropylamine, and tetrahydrofuran, etc.

The current collector is exemplified by copper, aluminum, titanium, stainless steel, nickel, calcined carbon material, a conductive polymer and conductive glass, etc.

<Electrolyte Solution>

The electrolyte solution of the invention contains the electrode protection film forming agent (D), an electrolyte (E) and a nonaqueous solvent (F), and is preferably useful as an electrolyte solution for lithium secondary batteries, for lithium-ion capacitors and for electrical double-layer capacitors.

The electrolyte solution of the invention contains the electrode protection film forming agent (D), the electrolyte (E) and the nonaqueous solvent (F) before being charged/discharged in use. As a charge/discharge process starts, a part of (D) undergoes a polymerization reaction to form a polymer film on the surface of the active material (Q) constituting the electrode. As the polymerization reaction proceeds, (D) in the electrolyte solution of the invention decreases.

As the electrolyte (E) for lithium secondary batteries and for lithium-ion capacitors, one commonly used in an electrolyte solution for lithium secondary batteries and lithium-ion capacitors can be used. Examples thereof include lithium salts of inorganic acids, such as LiPF₆, LiBF₄, LiSbF₆, LiAsF₆ and LiClO₄, etc., and lithium salts of organic acids, such as LiN(CF₃SO₂)₂, LiN(C₂F₅SO₂)₂ and LiC(CF₃SO₂)₃, etc. Among them, in view of the battery output and charge/discharge cycle characteristic, LiPF₆ is preferred.

As the electrolyte (E) for electrical double-layer capacitors, one used in a common electrolyte solution for electrical double-layer capacitors can be used. Examples thereof include: tetraalkylammonium salts, such as tetraethylammonium tetrafluoroborate salt, and triethylmethylammonium tetrafluoroborate salt, etc.; and amidinium salts, such as 1-ethyl-3-methylimidazolium tetrafluoroborate salt, etc.

As the nonaqueous solvent (F), one used in a common electrolyte solution for lithium secondary batteries, lithium-ion capacitors and electrical double-layer capacitors can be used. For example, a lactone compound, a cyclic or chain carbonate ester, a chain carboxylate ester, a cyclic or chain ether, a phosphate ester, a nitrile compound, an amide compound, a sulfone, sulfolane, etc. or a mixture thereof can be used.

Among the nonaqueous solvents (F), in view of the battery output and charge/discharge cycle characteristic, a cyclic or chain carbonate ester is preferred.

Specific examples of the cyclic carbonate ester include propylene carbonate, ethylene carbonate and butylene carbonate, etc.

Specific examples of the chain carbonate ester include dimethyl carbonate, methylethyl carbonate, diethyl carbonate, methyl n-propyl carbonate, ethyl n-propyl carbonate, and di n-propyl carbonate, etc.

Based on the total weight of the electrode protection film forming agent (D), the electrolyte (E) and the nonaqueous solvent (F) in the electrolyte solution of the invention, preferred contents of the electrode protection film forming agent (D), the electrolyte (E) and the nonaqueous solvent (F) are respectively described as follows.

In view of the charge/discharge cycle characteristic, battery capacity and high-temperature storage characteristic, the content of (D) is preferably 0.01 to 10 wt %, and more preferably 0.05 to 1 wt %.

In view of the battery output and charge/discharge cycle characteristic, the content of the electrolyte (E) in the electrolyte solution is preferably 0.1 to 30 wt %, and more preferably 0.5 to 20 wt %.

In view of the battery output and charge/discharge cycle characteristic, the content of the nonaqueous solvent (F) is preferably 60 to 99 wt %, and more preferably 85 to 95 wt %.

The electrolyte solution of the invention may further contain an additive such as an overcharge inhibitor, a dehydrating agent and a capacity stabilizer, etc. The content of each component in the additive described below is based on the total weight of the electrode protection film forming agent (D), the electrolyte (E) and the nonaqueous solvent (F).

The overcharge inhibitor is exemplified by an aromatic compound such as biphenyl, alkylbiphenyl, terphenyl, a partially hydrogenated product of terphenyl, cyclohexylbenzene, t-butylbenzene and t-amylbenzene, etc., or the like. The amount of the overcharge inhibitor used is generally 0 to 5 wt %, and preferably 0.5 to 3 wt %.

The dehydrating agent is exemplified by zeolite, silica gel and calcium oxide, etc. The amount of the dehydrating agent used is generally 0 to 5 wt %, and preferably 0.5 to 3 wt %, based on the overall weight of the electrolyte solution.

The capacity stabilizer is exemplified by fluoroethylene carbonate, succinic anhydride, 1-methyl-2-piperidone, heptane and fluorobenzene, etc. The amount of the capacity stabilizer used is generally 0 to 5 wt %, and preferably 0.5 to 3 wt %, based on the overall weight of the electrolyte solution.

The lithium secondary battery of the invention is obtained by using the electrode of the invention as a positive electrode or a negative electrode, or by using the electrolyte solution of the invention as the electrolyte solution, or by a combined use of both, in injecting the electrolyte solution in a battery can that accommodates the positive electrode, the negative electrode and a separator and sealing the battery can.

The separator in the lithium secondary battery is exemplified by a microporous film of a polyethylene or polypropylene film, a multilayer film of a porous polyethylene film and polypropylene, nonwoven fabric including polyester fiber, aramid fiber or glass fiber, etc. as well as a film obtained by attaching ceramic fine particles of silica, alumina or titania, etc. to the surfaces of the aforementioned.

The battery can in the lithium secondary battery can include a metal material such as stainless steel, iron, aluminum or nickel-plated steel, etc., but may alternatively include a plastic material, depending on use of the battery. Also, the battery can is of a cylindrical type, a coin type or a square type, or can be formed in any other shape, depending on the use.

A lithium-ion capacitor of the invention is obtained by, in a basic structure of the lithium secondary battery of the invention, replacing the positive electrode with a positive electrode for lithium-ion capacitors and replacing the battery can with a capacitor can. The material and shape of the capacitor can are, for example, the same as those exemplified with respect to the battery can.

The electrical double-layer capacitor of the invention is obtained by, in a basic structure of the lithium-ion capacitor of the invention, replacing the negative electrode with a negative electrode for electrical double-layer capacitors.

In the method for fabricating an electrode protection film of the invention, the electrode of the invention is used as a positive electrode or a negative electrode, or the electrolyte solution of the invention is used as an electrolyte solution, or both are used in combination, and then a voltage is applied thereto.

### EXAMPLES

The invention is further described below with examples, but is not limited thereto. Unless particularly defined, “%” means “wt %” and “part” means “weight part,” hereinafter.

Electrode Protection Film Forming Agent (D)

The number average molecular weight of a compound (C-15) was measured by GPC under the following conditions.

Device (as an example): HLC-8120 made by Tosoh Corporation;

Column (as an example): TSK GEL GMH6, 2 columns (made by Tosoh Corporation);

Measurement temperature: 40° C.

Sample solution: 0.25 wt % THF solution;

Solution injection amount: 100 μl;

Detector: Refractive index detector;

Reference material: Standard polystyrene (TSKstandard POLY STYRENE) produced by Tosoh Corporation, 5 points (Mw 500, 1050, 2800, 5970 and 9100).

### Preparation Example 1

### Synthesis of 1-hydroxymethyl-4-(1-propenoxymethyl)cyclohexane

9.86 parts of 1,4-cyclohexanedimethanol (by Tokyo Chemical Industry Co., Ltd.), 5.76 parts of allyl chloride (Tokyo Chemical Industry Co., Ltd.), 6.00 parts of sodium hydroxide and 100 parts of toluene were placed in a flask equipped with a stirrer, a thermometer and a cooling pipe, stirred while uniformly dissolved, then stirred at room temperature for 15 min, and then 1.32 parts of tetrabutylammonium bromide were added. The temperature of the mixture was raised to 65° C. over 2 hours and the resultant was further stirred for 4 hours to carry out an etherification reaction and a rearrangement reaction. After cooling, 200 parts of water were added to the resultant, and an aqueous layer was separated therefrom. Further, an organic layer was washed with 200 parts of water. After the toluene was removed under reduced pressure (1.3 kPa), the reactant was purified by an alumina column (150 mesh, Brockman 1, standard grade, made by Aldrich) with hexane as an eluent to obtain 9.0 parts of 1-hydroxymethyl-4-(1-propenoxymethyl)cyclohexane (yield: 71%).

### Example 1

### Synthesis of Compound (C-1) for Electrode Protection Film Forming Agent

15.0 parts of 1-hydroxymethyl-4-(1-propenoxymethyl)cyclohexane, 7.3 parts of butyl isocyanate, 100 parts of toluene and 0.5 part of N,N,N′,N′-tetramethylethylenediamine were placed in a flask equipped with a stirrer, a thermometer and a cooling pipe, and heated at 80° C. for 8 hours. After toluene was removed under reduced pressure (1.3 kPa), the reactant was purified by an alumina column (150 mesh, Brockman 1, standard grade, made by Sigma-Aldrich) with hexane and ethyl acetate as an eluent, to obtain 8.8 parts of compound (C-1) represented by the formula below [yield: 42%, Mn: 283 (calculated from the chemical formula)]. Compound (C-1) was used as an electrode protection film forming agent (D-1).

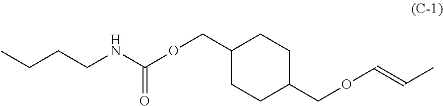 [SMILES: C/C=C/OCC1CCC(COC(=O)NCCCC)CC1]

### Example 2

### Synthesis of Compound (C-2) for Electrode Protection Film Forming Agent

Except that 6.5 parts of hexamethylenediisocyanate were used instead of 7.3 parts of butyl isocyanate, 7.7 parts of compound (C-2) represented by the formula below were obtained in the same way as Example 1 [yield: 37%, Mn: 536 (calculated from the chemical formula)]. Compound (C-2) was used as an electrode protection film forming agent (D-2).

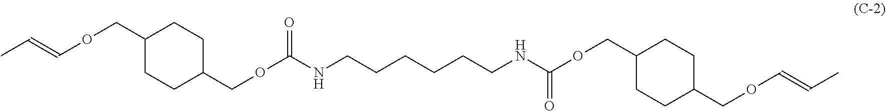 [SMILES: C/C=C/OCC1CCC(COC(=O)NCCCCCCNC(=O)OCC2CCC(CO/C=C/C)CC2)CC1]

### Example 3

### Synthesis of Compound (C-3) for Electrode Protection Film Forming Agent

Except that 10.0 parts of dicyclohexylmethane-4,4′-diisocyanate were used instead of 7.3 parts of butyl isocyanate, 10.1 parts of compound (C-3) represented by the formula below were obtained in the same way as Example 1 [yield: 40%, Mn: 630 (calculated from chemical formula)]. Compound (C-3) was used as an electrode protection film forming agent (D-3).

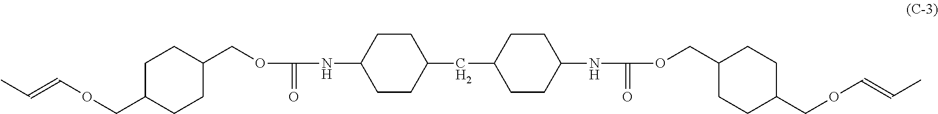 [SMILES: C/C=C/OCC1CCC(COC(=O)NC2CCC(CC3CCC(NC(=O)OCC4CCC(CO/C=C/C)CC4)CC3)CC2)CC1]

### Example 4

### Synthesis of Compound (C-4) for Electrode Protection Film Forming Agent

Except that 8.6 parts of isophorone didiisocyanate were used instead of 7.3 parts of butyl isocyanate, 10.1 parts of compound (C-4) represented by the formula below were obtained in the same way as Example 1 [yield: 44%, Mn: 590 (calculated from the chemical formula)]. Compound (C-4) was used as an electrode protection film forming agent (D-4).

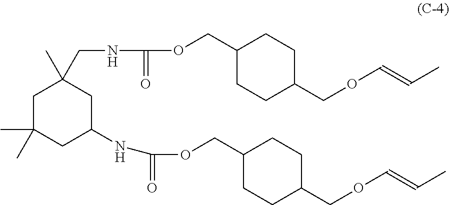 [SMILES: C/C=C/OCC1CCC(COC(=O)NCC2(C)CC(NC(=O)OCC3CCC(CO/C=C/C)CC3)CC(C)(C)C2)CC1]

### Example 5

### Synthesis of Compound (C-5) for Electrode Protection Film Forming Agent

Except that 9.7 parts of diphenylmethane diisocyanate were used instead of 7.3 parts of butyl isocyanate, 8.7 parts of compound (C-5) represented by the formula below were obtained in the same way as Example 1 [yield: 35%, Mn: 618 (calculated from the chemical formula)]. Compound (C-5) was used as an electrode protection film forming agent (D-5).

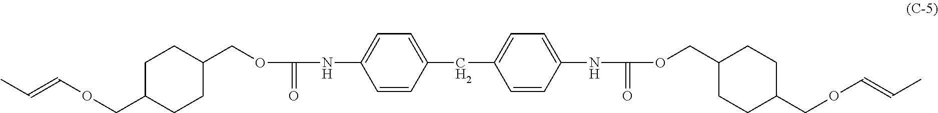 [SMILES: C/C=C/OCC1CCC(COC(=O)Nc2ccc(Cc3ccc(NC(=O)OCC4CCC(CO/C=C/C)CC4)cc3)cc2)CC1]

### Example 6

### Synthesis of Compound (C-6) for Electrode Protection Film Forming Agent

Except that 6.7 parts of toluene diisocyanate were used instead of 7.3 parts of butyl isocyanate, 8.8 parts of compound (C-6) represented by the formula below were obtained in the same way as Example 1 [yield: 42%, Mn: 542 (calculated from the chemical formula)]. Compound (C-6) was used as an electrode protection film forming agent (D-6).

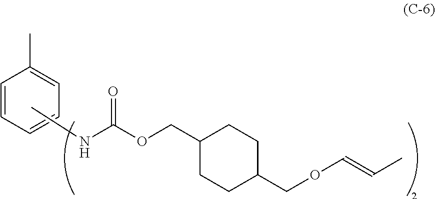

### Example 7

### Synthesis of Compound (C-7) for Electrode Protection Film Forming Agent

Except that 13.3 parts of a trimer of hexamethylenediisocyanate were used instead of 7.3 parts of butyl isocyanate, 9.3 parts of compound (C-7) represented by the formula below were obtained in the same way as Example 1 [yield: 33%, Mn: 1056 (calculated from the chemical formula)]. Compound (C-7) was used as an electrode protection film forming agent (D-7).

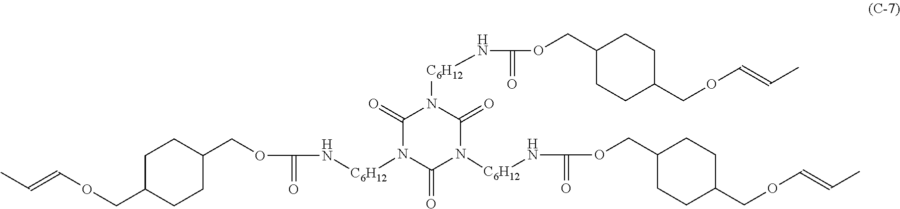

### Example 8

### Synthesis of Compound (C-8) for Electrode Protection Film Forming Agent

Except that 12.5 parts of linalool (produced by Wako Pure Chemical Industries, Ltd.) were used instead of 15.0 parts of 1-hydroxymethyl-4-(1-propenoxymethyl)cyclohexane, 9.7 parts of compound (C-8) represented by the formula below were obtained in the same way as Example 3 [yield: 45%, Mn: 570 (calculated from the chemical formula)]. Compound (C-8) was used as an electrode protection film forming agent (D-8).

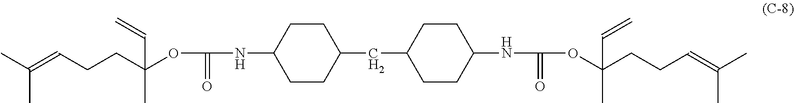 [SMILES: C=CC(C)(CCC=C(C)C)OC(=O)NC1CCC(CC2CCC(NC(=O)OC(C)(C=C)CCC=C(C)C)CC2)CC1]

### Example 9

### Synthesis of Compound (C-9) for Electrode Protection Film Forming Agent

Except that 12.5 parts of citronellol (produced by Wako Pure Chemical Industries, Ltd.) were used instead of 15.0 parts of 1-hydroxymethyl-4-(1-propenoxymethyl)cyclohexane, 10.1 parts of compound (C-9) represented by the formula below were obtained in the same way as Example 3 [yield: 47%, Mn: 574 (calculated from the chemical formula)]. Compound (C-9) was used as an electrode protection film forming agent (D-9).

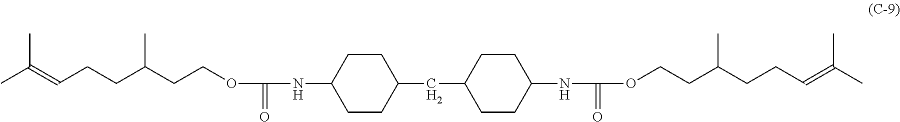 [SMILES: CC(C)=CCCC(C)CCOC(=O)NC1CCC(CC2CCC(NC(=O)OCCC(C)CCC=C(C)C)CC2)CC1]

### Example 10

### Synthesis of Compound (C-10) for Electrode Protection Film Forming Agent

Except that 12.5 parts of geraniol (produced by Wako Pure Chemical Industries, Ltd.) were used instead of 15.0 parts of 1-hydroxymethyl-4-(1-propenoxymethyl)cyclohexane, 12.2 parts of compound (C-10) represented by the formula below were obtained in the same way as Example 3 [yield: 56%, Mn: 570 (calculated from the chemical formula)]. Compound (C-10) was used as an electrode protection film forming agent (D-10).

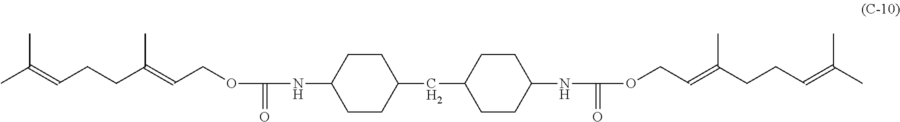 [SMILES: CC(C)=CCC/C(C)=C/COC(=O)NC1CCC(CC2CCC(NC(=O)OC/C=C(\C)CCC=C(C)C)CC2)CC1]

### Example 11

### Synthesis of Compound (C-11) for Electrode Protection Film Forming Agent

Except that 9.3 parts of 2-hydroxyethyl acrylate (by Wako Pure Chemical Industries, Ltd.) were used instead of 15.0 parts of 1-hydroxymethyl-4-(1-propenoxymethyl)cyclohexane, 9.5 parts of compound (C-11) represented by the formula below were obtained in the same way as Example 3 [yield: 50%, Mn: 494 (calculated from the chemical formula)]. Compound (C-11) was used as an electrode protection film forming agent (D-11).

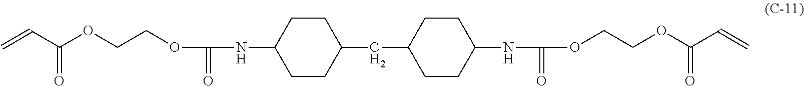 [SMILES: C=CC(=O)OCCOC(=O)NC1CCC(CC2CCC(NC(=O)OCCOC(=O)C=C)CC2)CC1]

### Example 12

### Synthesis of Compound (C-12) for Electrode Protection Film Forming Agent

Except that 10.8 parts of cinnamyl alcohol (by Wako Pure Chemical Industries, Ltd.) were used instead of 15.0 parts of 1-hydroxymethyl-4-(1-propenoxymethyl)cyclohexane, 8.5 parts of compound (C-12) represented by the formula below were obtained in the same way as Example 3 [yield: 42%, Mn: 530 (calculated from the chemical formula)]. Compound (C-12) was used as an electrode protection film forming agent (D-12).

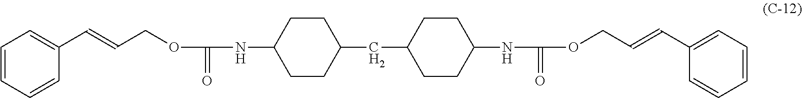 [SMILES: O=C(NC1CCC(CC2CCC(NC(=O)OC/C=C/c3ccccc3)CC2)CC1)OC/C=C/c1ccccc1]

### Example 13

### Synthesis of Compound (C-13) for Electrode Protection Film Forming Agent

Except that 11.9 parts of (E)-2-methyl-3-phenyl-2-propen-1-ol (by Tokyo Chemical Industry Co., Ltd.) were used instead of 15.0 parts of 1-hydroxymethyl-4-(1-propenoxy-methyl)cyclohexane, 8.3 parts of compound (C-13) represented by the formula below were obtained in the same way as Example 3 [yield: 39%, Mn: 558 (calculated from the chemical formula)]. Compound (C-13) was used as an electrode protection film forming agent (D-13).

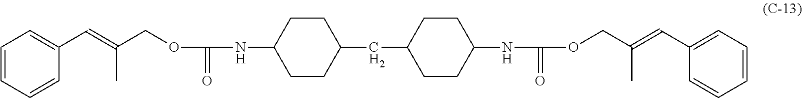 [SMILES: C/C(=C\c1ccccc1)COC(=O)NC1CCC(CC2CCC(NC(=O)OC/C(C)=C/c3ccccc3)CC2)CC1]

### Example 14

### Synthesis of Compound (C-14) for Electrode Protection Film Forming Agent

Except that 10.8 parts of (4-ethenylphenyl)methanol (by Tokyo Chemical Industry Co., Ltd.) were used instead of 15.0 parts of 1-hydroxymethyl-4-(1-propenoxymethyl)cyclohexane, 9.5 parts of compound (C-14) represented by the formula below were obtained in the same way as Example 3 [yield: 47%, Mn: 530 (calculated from chemical formula)]. Compound (C-14) was used as an electrode protection film forming agent (D-14).

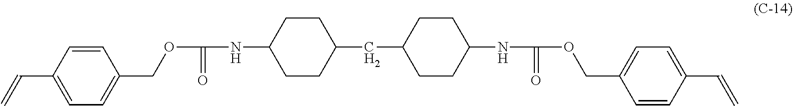 [SMILES: C=Cc1ccc(COC(=O)NC2CCC(CC3CCC(NC(=O)OCc4ccc(C=C)cc4)CC3)CC2)cc1]

### Example 15

### Synthesis of Compound (C-15) for Electrode Protection Film Forming Agent

5.5 parts of 1,4-cyclohexanedimethanol (by Tokyo Chemical Industry Co., Ltd.), 15.0 parts of dicyclohexylmethane-4,4′-diisocyanate, 100 parts of toluene and 0.5 part of N,N,N′,N′-tetramethylethylenediamine were placed in a flask equipped with a stirrer, a thermometer and a cooling pipe, and heated at 80° C. for 5 hours. Next, 5.9 parts of linalool (by Wako Pure Chemical Industries, Ltd.) were placed therein, and the mixture was heated at 80° C. for 5 hours. After the toluene was removed under a reduced pressure (1.3 kPa), the reactant was purified by a silica gel column (made by Wako Pure Chemical Industries, Ltd.) with hexane and ethyl acetate as an eluent to obtain 18.5 parts of compound (C-15) represented by the formula below [yield: 35%, Mn: 3,400 (result of GPC measurement)]. Compound (C-15) was used as an electrode protection film forming agent (D-15).

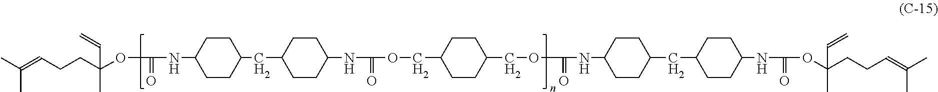

The electrode protection film forming agents (D-1) to (D-15) in Examples 1 to 15 are summarized in Table 1.

TABLE 1
Electrode protection film forming agent (D)

| | | | | | | Concentration | |
|---|---|---|---|---|---|---|---|
| | Electrode | | | | | of | |
| | protection | | | | Concentration | polymerizable | |
| | film | | Polyisocyanate | Compound (H) having | of urethane | unsaturated | |
| | forming | Compound | compound (G) having | polymerizable unsaturated | bond (a) | bond (b) | |
| Example | agent (D) | (C) | structure A | bond (b) | [mmol/g] | [mmol/g] | Mn |
| 1 | D-1 | C-1 | Butyl isocyanate | 1-hydroxymethyl-4- | 3.53 | 3.53 | 283 |
| | | | | (1-propenoxymethyl)cyclohexane | | | |
| 2 | D-2 | C-2 | Hexamethylenediisocyanate | 1-hydroxymethyl-4- | 3.73 | 3.73 | 536 |
| | | | | (1-propenoxymethyl)cyclohexane | | | |
| 3 | D-3 | C-3 | Dicyclohexylmethane-4,4′- | 1-hydroxymethyl-4- | 3.17 | 3.17 | 630 |
| | | | diisocyanate | (1-propenoxymethyl)cyclohexane | | | |
| 4 | D-4 | C-4 | Isophorone didiisocyanate | 1-hydroxymethyl-4- | 3.39 | 3.39 | 590 |
| | | | | (1-propenoxymethyl)cyclohexane | | | |
| 5 | D-5 | C-5 | Diphenylmethane diisocyanate | 1-hydroxymethyl-4- | 3.24 | 3.24 | 618 |
| | | | | (1-propenoxymethyl)cyclohexane | | | |
| 6 | D-6 | C-6 | Toluene diisocyanate | 1-hyclroxymethyl-4- | 3.69 | 3.69 | 542 |
| | | | | (1-propenoxymethyl)cyclohexane | | | |
| 7 | D-7 | C-7 | Trimer of | 1-hydroxymethyl-4- | 2.84 | 2.84 | 1056 |
| | | | hexamethylenediisocyanate | (1-propenoxymethyl)cyclohexane | | | |
| 8 | D-8 | C-8 | Dicyclohexylinethane- | Linalool | 3.51 | 7.02 | 570 |
| | | | 4,4′-diisocyanate | | | | |
| 9 | D-9 | C-9 | Dicyclohexylmethane- | Citronellol | 3.48 | 3.48 | 574 |
| | | | 4,4′-diisocyanate | | | | |
| 10 | D-10 | C-10 | Dicyclohexylmethane- | Geraniol | 3.51 | 7.02 | 570 |
| | | | 4,4′-diisocyanate | | | | |
| 11 | D-11 | C-11 | Dicyclohexylmethane- | 2-hydroxyethyl acrylate | 4.05 | 4.05 | 494 |
| | | | 4,4′-diisocyanate | | | | |
| 12 | D-12 | C-12 | Dicyclohexylmethane- | Cinnamyl alcohol | 3.77 | 3.77 | 530 |
| | | | 4,4′-diisocyanate | | | | |
| 13 | D-13 | C-13 | Dicyclohexylmethane- | (E)-2-methyl-3-phenyl-2- | 3.58 | 3.58 | 558 |
| | | | 4,4′-diisocyanate | propen-1-ol | | | |
| 14 | D-14 | C-14 | Dicyclohexylmethane- | (4-ethenylphenyl)methanol | 3.77 | 3.77 | 530 |
| | | | 4,4′-diisocyanate | | | | |
| 15 | D-15 | C-15 | Urethane prepolymer being a | Linalool | 4.69 | 1.18 | 3400 |
| | | | reaction product of | | | | |
| | | | dicyclohexylmethane-4,4′- | | | | |
| | | | diisocyanate and 1,4- | | | | |
| | | | cyclohexanedimethanol and having | | | | |
| | | | isocyanate groups at both ends | | | | |

The Mn in Examples 1 to 14 was a value calculated from the structural formula, and the Mn in Example 15 was a value measured by GPC.

### Examples 16 to 32 and Comparative Examples 1 to 3

### Evaluation of Lithium Secondary Batteries and Electrodes

Electrodes for lithium secondary batteries that contain the above-mentioned electrode protection film forming agent (D) or a comparative electrode protection film forming agent (D′) in a number of parts shown in Table 2 were fabricated by the method described later, and lithium secondary batteries were fabricated using the electrodes by the method described later.

Results of evaluation of the high-voltage charge/discharge cycle characteristic and the high-temperature storage characteristic by the methods described later are shown in Table 2.

TABLE 2
Evaluation Results of Lithium Secondary Batteries

| | | Electrode | | | | High-voltage | |
|---|---|---|---|---|---|---|---|
| | | protection film | Positive | Negative | Electrolyte | charge/discharge | High-temperature |
| | Comparative | forming agent | electrode | electrode | solution | cycle characteristic | storage |
| Example | Example | (D) | (part) | (part) | (part) | (%) | characteristic (% ) |
| 16 | — | D-1 | 0.5 | — | — | 93.7 | 95.4 |
| 17 | — | D-2 | 0.5 | — | — | 95.2 | 94.1 |
| 18 | — | D-3 | 0.5 | — | — | 97.3 | 98.2 |
| 19 | — | D-4 | 0.5 | — | — | 96.8 | 97.0 |
| 20 | — | D-5 | 0.5 | — | — | 93.2 | 94.1 |
| 21 | — | D-6 | 0.5 | — | — | 94.0 | 95.4 |
| 22 | — | D-7 | 0.5 | — | — | 92.8 | 93.5 |
| 23 | — | D-8 | 0.5 | — | — | 97.3 | 98.2 |
| 24 | — | D-9 | 0.5 | — | — | 96.8 | 97.0 |
| 25 | — | D-10 | 0.5 | — | — | 94.2 | 95.3 |
| 26 | — | D-11 | 0.5 | — | — | 92.6 | 93.3 |
| 27 | — | D-12 | 0.5 | — | — | 96.7 | 97.1 |
| 28 | — | D-13 | 0.5 | — | — | 98.0 | 95.7 |
| 29 | — | D-14 | 0.5 | — | — | 95.9 | 96.5 |
| 30 | — | D-15 | 0.5 | — | — | 96.6 | 95.9 |
| 31 | — | D-8 | — | 0.5 | — | 93.2 | 95.8 |
| 32 | — | D-9 | 0.5 | — | — | 95.1 | 94.9 |
| | — | D-10 | — | 0.5 | — | | |
| — | 1 | — | — | — | — | 72.5 | 60.7 |
| — | 2 | D'-1 | 0.5 | — | — | 84.3 | 67.2 |
| — | 3 | D'-2 | 0.5 | — | — | 79.8 | 72.0 |
| 33 | — | D-1 | — | — | 0.5 | 94.5 | 93.6 |
| 34 | — | D-2 | — | — | 0.5 | 95.0 | 97.2 |
| 35 | — | D-3 | — | — | 0.5 | 97.2 | 98.1 |
| 36 | — | D-4 | — | — | 0.5 | 96.6 | 95.8 |
| 37 | — | D-5 | — | — | 0.5 | 92.1 | 93.9 |
| 38 | — | D-6 | — | — | 0.5 | 92.5 | 95.5 |
| 39 | — | D-7 | — | — | 0.5 | 91.8 | 93.2 |
| 40 | — | D-8 | — | — | 0.5 | 93.8 | 92.6 |
| 41 | — | D-9 | — | — | 0.5 | 93.5 | 96.8 |
| 42 | — | D-10 | — | — | 0.5 | 97.2 | 96.2 |
| 43 | — | D-11 | — | — | 0.5 | 92.8 | 95.0 |
| 44 | — | D-12 | — | — | 0.5 | 94.6 | 94.9 |
| 45 | — | D-13 | — | — | 0.5 | 97.0 | 94.8 |
| 46 | — | D-14 | — | — | 0.5 | 94.5 | 92.7 |
| 47 | — | D-15 | — | — | 0.5 | 93.8 | 95.8 |
| — | 4 | — | — | — | — | 73.5 | 62.0 |
| — | 5 | D′-1 | — | — | 0.5 | 76.3 | 72.1 |
| — | 6 | D′-2 | — | — | 0.5 | 80.4 | 75.4 |

[Fabrication of Positive Electrode for Lithium Secondary Batteries]

90.0 parts of LiCoO₂ powder, 5 parts of Ketjen black (by Sigma-Aldrich), 5 parts of polyvinylidene fluoride (by Sigma-Aldrich), and (D) in a number of parts shown in Table 2 were sufficiently mixed together in a mortar, then 70.0 parts of 1-methyl-2-pyrrolidone (by Tokyo Chemical Industry Co., Ltd.) were added, and the above materials were further sufficiently mixed in the mortar to obtain a slurry. The obtained slurry was coated on one side of an aluminum electrolytic foil of 20 μm thick using a wire bar in the atmosphere, dried at 80° C. for 1 hour, then further dried at 80° C. for 2 hours under a reduced pressure (1.3 kPa), and punched into 15.95 mmφ, so as to fabricate a positive electrode for the lithium secondary batteries of Examples 16 to 32.

[Fabrication of Negative Electrode for Lithium Secondary Batteries]

92.5 parts of graphite powder having a mean particle size of about 8 to 12 μm, 7.5 parts of polyvinylidene fluoride, 200 parts of 1-methyl-2-pyrrolidone (by Tokyo Chemical Industry Co., Ltd.), and (D) in a number of parts shown in Table 2 were sufficiently mixed together in a mortar to obtain a slurry. The obtained slurry was coated on one side of a copper foil of 20 μm thick using a wire bar in the atmosphere, dried at 80° C. for 1 hour, then further dried at 80° C. for 2 hours under reduced pressure (1.3 kPa), punched into 16.15 mmφ and formed into 30 μm in thickness by means of a press machine, so as to fabricate a negative electrode for the lithium secondary batteries of Examples 16 to 32.

### Comparative Example 1

Except that the electrode protection film forming agent (D) was not added, the negative electrode and the positive electrode for the lithium secondary battery of Comparative Example 1 were fabricated with the same methods as in Example 16.

### Comparative Example 2

Except that 0.5 part of methyl phenyl sulfide (D′-1) as a comparative additive was added instead of the electrode protection film forming agent (D), the negative electrode and the positive electrode for the lithium secondary battery of Comparative Example 2 were fabricated with the same methods as in Example 16.

### Comparative Example 3

Except that 0.5 part of diphenyl sulfide (D′-2) was added as a comparative additive instead of the electrode protection film forming agent (D), the negative electrode and the positive electrode for the lithium secondary battery of Comparative Example 3 were fabricated with the same methods as in Example 16.

[Fabrication of Lithium Secondary Battery]

The positive electrodes and the negative electrodes of Examples 16 to 32 and Comparative Examples 1 to 3 were arranged on both ends of a 2032-type coin cell with the respective coated surfaces facing each other, and a separator (a nonwoven fabric made from polypropylene) was inserted between the electrodes, so as to fabricate a cell for a lithium secondary battery. An electrolyte solution obtained by dissolving LiPF₆ in a mixed solvent of ethylene carbonate (EC) and diethyl carbonate (DEC) (volume ratio: 1:1) in a ratio of 12 wt % was injected into the fabricated cell and sealed. Results of evaluation of the high-voltage charge/discharge cycle characteristic and high-temperature storage characteristic by the following methods are shown in Table 2.

<Evaluation of High-Voltage Charge/Discharge Cycle Characteristic>

By using the charge/discharge tester “Battery Analyzer 1470” (made by Toyo Corporation), a charging was performed at a current of 0.1 C until a voltage of 4.5 V was reached, and after a 10-minute pause, a discharge was performed at a current of 0.1 C until the battery voltage reached 3.5 V. This charge/discharge process was repeated. At this moment, the battery capacity at the initial charge and the battery capacity at the charge of the 50^{th} cycle were measured, from which the charge/discharge cycle characteristic was calculated using the following equation. A greater numerical value means a better charge/discharge cycle characteristic.

High-voltage charge/discharge cycle characteristic (%)=(battery capacity at the charge of the 50^{th} cycle/battery capacity at the initial charge)×100

<Evaluation of High-Temperature Storage Characteristic>

By using the charge/discharge tester “Battery Analyzer 1470” (by Toyo Corporation), a charging was performed at a current of 0.1 C until a voltage of 4.5 V was reached, and after a 10-minute pause, a discharging was performed at a current of 0.1 C until a voltage of 3.5 V was reached, and the capacity was measured (initial battery capacity). Further, a charging was performed at a current of 0.1 C until a voltage of 4.5 V was reached, and after a 7-day storage at 85° C., a discharge was performed at a current of 0.1 C until a voltage of 3.5 V was reached, and the battery capacity was measured (battery capacity after high-temperature storage), from which the high-temperature storage characteristic was calculated using the following equation. A greater numerical value means a better high-temperature storage characteristic.

High-temperature storage characteristic (%)=(battery capacity after high-temperature storage/initial battery capacity)×100

### Examples 33 to 47 and Comparative Examples 4 to 6

### Evaluation of Lithium Secondary Batteries and Electrolyte Solutions

Lithium secondary batteries that used electrolyte solutions containing the above electrode protection film forming agent (D) or comparative electrode protection film forming agent (D′) in a number of parts shown in Table 2 were fabricated by the following method.

Similar to the case of the electrodes, the high-voltage charge/discharge cycle characteristic and the high-temperature storage characteristic were evaluated by the above-mentioned methods, and results thereof are shown in Table 2.

[Preparation of Electrolyte Solution]

The electrode protection film forming agent (D) in a number of parts shown in Table 2 was compounded into 87.5 parts of a mixed solvent of EC and DEC (volume ratio: 1:1), and LiPF₆ as the electrolyte (E) was dissolved therein so that the content of LiPF₆ is 12 wt %, thereby preparing an electrolyte solution of Examples 33 to 47.

### Comparative Example 4

Except that the electrode protection film forming agent (D) was not added, the electrolyte solution of Comparative Example 4 was prepared by the same method of Example 33.

### Comparative Example 5

Except that 0.5 part of methyl phenyl sulfide (D′-1) as a comparative additive was added instead of the electrode protection film forming agent (D), the electrolyte solution of Comparative Example 5 was prepared by the same method of Example 33.

### Comparative Example 6

Except that 0.5 part of diphenyl sulfide (D′-2) as a comparative additive was added instead of the electrode protection film forming agent (D), the electrolyte solution of Comparative Example 6 was prepared by the same method of Example 33.

[Fabrication of Positive Electrode for Lithium Secondary Batteries]

90.0 parts of LiCoO₂ powder, 5 parts of Ketjen black (by Sigma-Aldrich), and 5 parts of polyvinylidene fluoride (by Sigma-Aldrich) were sufficiently mixed together in a mortar, then 70.0 parts of 1-methyl-2-pyrrolidone (by Tokyo Chemical Industry Co., Ltd.) were added, and the above materials were further sufficiently mixed in the mortar to obtain a slurry. The obtained slurry was coated on one side of an aluminum electrolytic foil of 20 μm thick using a wire bar in the atmosphere, dried at 80° C. for 1 hr, then further dried at 80° C. for 2 hr under a reduced pressure (1.3 kPa), and punched into 15.95 mmφ, so as to fabricate a positive electrode for lithium secondary batteries.

[Fabrication of Negative Electrode for Lithium Secondary Batteries]

92.5 parts of graphite powder having a mean particle size of about 8 to 12 μm, 7.5 parts of polyvinylidene fluoride and 200 parts of 1-methyl-2-pyrrolidone were sufficiently mixed together in a mortar to obtain a slurry. The obtained slurry was coated on one side of a copper foil of 20 μm thick using a wire bar in the atmosphere, dried at 80° C. for 1 hour, then further dried at 80° C. for 2 hours under reduced pressure (1.3 kPa), punched into 16.15 mmφ and formed into 30 μm in thickness by means of a pressing machine, so as to fabricate a graphite-based negative electrode for lithium secondary batteries.

[Fabrication of Secondary Battery]

The above-mentioned positive electrode and negative electrode were arranged on both ends of a 2032-type coin cell with the respective coated surfaces facing each other, and a separator (a nonwoven fabric made from polypropylene) was inserted between the electrodes, so as to fabricate a cell for lithium secondary batteries.

The electrolyte solutions of Examples 33 to 47 and Comparative Examples 4 to 6 were respectively injected into the fabricated cells for lithium secondary batteries and then sealed, thereby fabricating secondary batteries.

### Examples 48 to 64 and Comparative Examples 7 to 9

### Evaluation of Lithium-Ion Capacitors and Electrodes

Electrodes for Li-ion capacitors that contained the above electrode protection film forming agent (D) or comparative electrode protection film forming agent (D′) in a number of parts shown in Table 3 were fabricated by the method described later, and Li-ion capacitors were fabricated using the electrode by the method described later.

Results of evaluation of the high-voltage charge/discharge cycle characteristic and the high-temperature storage characteristic by the methods described later are shown in Table 3.

TABLE 3
Evaluation Results of Lithium-ion Capacitors

| | | Electrode | | | | High-voltage | |
|---|---|---|---|---|---|---|---|
| | | protection film | Positive | Negative | Electrolyte | charge/discharge | High-temperature |
| | Comparative | forming agent | electrode | electrode | solution | cycle characteristic | storage |
| Example | Example | (D) | (part) | (part) | (part) | (%) | characteristic (% ) |
| 48 | — | D-1 | 0.5 | — | — | 95.0 | 93.6 |
| 49 | — | D-2 | 0.5 | — | — | 96.1 | 94.4 |
| 50 | — | D-3 | 0.5 | — | — | 96.9 | 97.5 |
| 51 | — | D-4 | 0.5 | — | — | 97.2 | 96.0 |
| 52 | — | D-5 | 0.5 | — | — | 92.8 | 93.0 |
| 53 | — | D-6 | 0.5 | — | — | 93.5 | 94.1 |
| 54 | — | D-7 | 0.5 | — | — | 92.3 | 92.1 |
| 55 | — | D-8 | 0.5 | — | — | 97.0 | 92.4 |
| 56 | — | D-9 | 0.5 | — | — | 93.3 | 97.2 |
| 57 | — | D-10 | 0.5 | — | — | 95.7 | 95.5 |
| 58 | — | D-11 | 0.5 | — | — | 94.6 | 91.8 |
| 59 | — | D-12 | 0.5 | — | — | 95.7 | 93.0 |
| 60 | — | D-13 | 0.5 | — | — | 97.2 | 94.3 |
| 61 | — | D-14 | 0.5 | — | — | 94.8 | 95.2 |
| 62 | — | D-15 | 0.5 | — | — | 93.6 | 96.1 |
| 63 | — | D-8 | — | 0.5 | — | 94.2 | 97.7 |
| 64 | — | D-9 | 0.5 | — | — | 98.5 | 94.5 |
| | — | D-10 | — | 0.5 | — | | |
| — | 7 | — | — | — | — | 76.2 | 54.0 |
| — | 8 | D′-1 | 0.5 | — | — | 80.3 | 67.7 |
| — | 9 | D′-2 | 0.5 | — | — | 82.6 | 62.4 |
| 65 | — | D-1 | — | — | 0.5 | 95.3 | 94.8 |
| 66 | — | D-2 | — | — | 0.5 | 96.2 | 94.0 |
| 67 | — | D-3 | — | | 0.5 | 98.2 | 96.9 |
| 68 | — | D-4 | — | — | 0.5 | 96.8 | 97.2 |
| 69 | — | D-5 | — | — | 0.5 | 92.2 | 93.9 |
| 70 | — | D-6 | — | — | 0.5 | 93.4 | 95.4 |
| 71 | — | D-7 | — | — | 0.5 | 92.3 | 92.9 |
| 72 | — | D-8 | — | — | 0.5 | 97.2 | 96.4 |
| 73 | — | D-9 | — | — | 0.5 | 95.1 | 93.1 |
| 74 | — | D-10 | — | — | 0.5 | 93.8 | 95.6 |
| 75 | — | D-11 | — | — | 0.5 | 94.5 | 92.1 |
| 76 | — | D-12 | — | — | 0.5 | 95.2 | 95.5 |
| 77 | — | D-13 | — | — | 0.5 | 92.5 | 94.2 |
| 78 | — | D-14 | — | — | 0.5 | 96.8 | 96.7 |
| 79 | — | D-15 | — | — | 0.5 | 94.3 | 95.1 |
| — | 10 | — | — | — | — | 75.4 | 55.6 |
| — | 11 | D′-1 | — | — | 0.5 | 82.1 | 60.0 |
| — | 12 | D′-2 | — | — | 0.5 | 77.3 | 64.9 |

[Fabrication of Positive Electrode for Lithium-Ion Capacitors]

90.0 parts of activated carbon powder, 5.0 parts of Ketjen black (by Sigma-Aldrich), 5.0 parts of polyvinylidene fluoride (by Sigma-Aldrich), and (D) in a number of parts shown in Table 3 were sufficiently mixed together in a mortar, then 70.0 parts of 1-methyl-2-pyrrolidone (by Tokyo Chemical Industry Co., Ltd.) were added, and the resultant above materials were further sufficiently mixed in the mortar to obtain a slurry. The obtained slurry was coated on one side of an aluminum electrolytic foil of 20 μm thick using a wire bar in the atmosphere, dried at 80° C. for 1 hr, then further dried at 80° C. for 2 hr under a reduced pressure (1.3 kPa), and punched into 15.95 mmφ, so as to fabricate a positive electrode for lithium-ion capacitors.

[Fabrication of Negative Electrode for Lithium-Ion Capacitors]

92.5 parts of graphite powder having a mean particle size of about 8 to 12 μm, 7.5 parts of polyvinylidene fluoride, 200 parts of 1-methyl-2-pyrrolidone (by Tokyo Chemical Industry Co., Ltd.), and (D) in a number of parts shown in Table 3 were sufficiently mixed together in a mortar to obtain a slurry. The obtained slurry was coated on one side of a copper foil of 20 μm thick, dried at 80° C. for 1 hr, then further dried at 80° C. for 2 hr under a reduced pressure (1.3 kPa), punched into 16.15 mmφ and formed into 30 μm in thickness by means of a press machine. The obtained electrode and a lithium metal foil were sandwiched by a separator (nonwoven fabric made from polypropylene) and set in a beaker cell, and lithium ions in an amount of about 75% of the theoretical capacity of the negative electrode were occluded into the negative electrode over ˜10 hours, so as to fabricate a negative electrode for Li-ion capacitors.

### Comparative Example 7

Except that the electrode protection film forming agent (D) was not added, the negative electrode and the positive electrode for the Li-ion capacitor of Comparative Example 7 were fabricated with the same method of Example 48.

### Comparative Example 8

Except that 0.5 part of methyl phenyl sulfide (D′-1) as a comparative additive was added instead of the electrode protection film forming agent (D), the negative electrode and the positive electrode for the Li-ion capacitor of Comparative Example 8 were fabricated with the same method of Example 48.

### Comparative Example 9

Except that 0.5 part of diphenyl sulfide (D′-2) as a comparative additive was added instead of the electrode protection film forming agent (D), the negative electrode and the positive electrode for the Li-ion capacitor of Comparative Example 9 were fabricated with the same method of Example 48.

[Fabrication of Li-Ion Capacitor]

The positive electrode and the negative electrode of each of Examples 48 to 64 and Comparative Examples 7 to 9 were arranged in a storage case including an aluminum laminated polypropylene film with the respective coated surfaces facing each other, and a separator (nonwoven fabric made from polypropylene) was inserted between the electrodes, so as to fabricate a cell for capacitors. An electrolyte solution obtained by dissolving LiPF₆ in propylene carbonate (PC) in a proportion of 12 wt % was injected into the fabricated cell and sealed.

<Evaluation of High-Voltage Charge/Discharge Cycle Characteristic>

By using the charge/discharge tester “Battery Analyzer 1470” (made by Toyo Corporation), a charging was performed at a current of 1 C until a voltage of 3.8 V was reached, and after a 10-minute pause, a discharging was performed at a current of 1 C until a voltage of 2.0 V was reached. This charge/discharge process was repeated. At this moment, the battery capacity at the initial charge and the battery capacity at the charge of the 50^{th} cycle were measured, from which the charge/discharge cycle characteristic was calculated using the following equation. A greater numerical value means a better charge/discharge cycle characteristic.

High-voltage charge/discharge cycle characteristic (%)=(battery capacity at the charge of the 50^{th} cycle/battery capacity at the initial charge)×100

<Evaluation of High-Temperature Storage Characteristic>

By using the charge/discharge tester “Battery Analyzer 1470” (by Toyo Corporation), a charging was performed at a current of 1 C until a voltage of 3.8 V was reached, and after a 10-minute pause, a discharging was performed at a current of 1 C until a voltage of 2.0 V was reached, and the capacity was measured (initial battery capacity). Further, a charging was performed at a current of 1 C until a voltage of 3.8 V was reached, and after a 7-day storage at 85° C., a discharge was performed at a current of 1 C until a voltage of 2.0 V was reached, and the battery capacity was measured (battery capacity after high-temperature storage), from which the high-temperature storage characteristic was calculated with the following equation. A greater numerical value means a better high-temperature storage characteristic.

High-temperature storage characteristic (%)=(battery capacity after high-temperature storage/initial battery capacity)×100

### Examples 65 to 79 and Comparative Examples 10 to 12

### Evaluation of Lithium-Ion Capacitors and Electrolyte Solutions

Li-ion capacitors each using an electrolyte solution containing the above electrode protection film forming agent (D) or comparative electrode protection film forming agent (D′) in a number of parts shown in Table 3 were fabricated using the method described later.

Similar to the case of the electrodes, results of evaluation of the high-voltage charge/discharge cycle characteristic and the high-temperature storage characteristic by the above-mentioned method are shown in Table 3.

[Preparation of Electrolyte Solution]

The electrode protection film forming agent (D) in a number of parts shown in Table 3 was compounded into the nonaqueous solvent (F) including 87.5 parts of PC, and LiPF₆ as the electrolyte (E) was dissolved therein so that the content of LiPF₆ is 12 wt %, thereby preparing the electrolyte solutions of Examples 65 to 79.

### Comparative Example 10

Except that the electrode protection film forming agent (D) was not added, the electrolyte solution of Comparative Example 10 was prepared by the same method of Example 65.

### Comparative Example 11

Except that 0.5 part of methyl phenyl sulfide (D′-1) as a comparative additive was added instead of the electrode protection film forming agent (D), the electrolyte solution of Comparative Example 11 was prepared by the same method of Example 65.

### Comparative Example 12

Except that 0.5 part of diphenyl sulfide (D′-2) as a comparative additive was added instead of the electrode protection film forming agent (D), the electrolyte solution of Comparative Example 12 was prepared by the same method of Example 65.

[Fabrication of Positive Electrode]

Activated carbon having a specific surface area of ˜2200 m²/g that was obtained with an alkali activation method was used as a positive electrode active material. Activated carbon powder, acetylene black and polyvinylidene fluoride were mixed together so that a weight ratio between them is 80:10:10. This mixture was added to 1-methyl-2-pyrrolidone as a solvent, stirred and mixed together to obtain a slurry. This slurry was coated on an aluminum foil of 30 μm thick by a doctor blade method, then preliminarily dried, and then cut out to obtain an electrode having a size of 20 mm×30 mm. The electrode was about 50 μm in thickness. Before the assembly of the cell, the electrode was dried at 120° C. for 10 hours in vacuum, so as to fabricate a positive electrode for Li-ion capacitors.

[Fabrication of Negative Electrode]

80 parts of graphite powder having a mean particle size of about 8 to 12 μm, 10 parts of acetylene black and 10 parts of polyvinylidene fluoride were mixed together, and this mixture was added to 1-methyl-2-pyrrolidone as a solvent, stirred and mixed together to obtain a slurry. This slurry was coated on a copper foil of 18 μm thick by a doctor blade method, then preliminarily dried, and then cut out to obtain an electrode having a size of 20 mm×30 mm. The electrode was about 50 μm in thickness. The electrode was further dried at 120° C. for 5 hours in vacuum. The obtained electrode and a lithium metal foil were sandwiched by a separator (nonwoven fabric made from polypropylene) and set in a beaker cell, and Li-ions in an amount of about 75% of theoretical capacity of the negative electrode were occluded into the negative electrode over about 10 hours, so as to fabricate a negative electrode for Li-ion capacitors.

[Assembly of Capacitor Cell]

A separator (nonwoven fabric made from polypropylene) was inserted between the above-mentioned positive electrode and negative electrode. The resultant was impregnated with an electrolytic solutions of Examples 65 to 79 and Comparative Examples 10 to 12, placed into a storage case including an aluminum laminated polypropylene film and sealed, so as to fabricate a Li-ion capacitor cell.

### Examples 80 to 96 and Comparative Examples 13 to 15

### Evaluation of Electrical Double-Layer Capacitors and Electrodes

A positive electrode and a negative electrode for electrical double-layer capacitors that contain the above-mentioned electrode protection film forming agent (D) or comparative electrode protection film forming agent (D′) in a number of parts shown in Table 4 were fabricated with the method described later, and an electrical double-layer capacitor was fabricated using the electrodes with the method described later.

Results of evaluation of the long-term durability by the method described later are shown in Table 4.

TABLE 4
Evaluation Results of Electrical Double-layer Capacitors

| | | Electrode | Positive | Negative | Electrolyte | Capacity |
|---|---|---|---|---|---|---|
| | Comparative | protection film | electrode | electrode | solution | retention ratio |
| Example | Example | forming agent (D) | (part) | (part) | (part) | (%) |
| 80 | — | D-1 | 0.5 | — | — | 92.7 |
| 81 | — | D-2 | 0.5 | — | — | 93.6 |
| 82 | — | D-3 | 0.5 | — | — | 94.8 |
| 83 | — | D-4 | 0.5 | — | — | 94.4 |
| 84 | — | D-5 | 0.5 | — | — | 95.4 |
| 85 | — | D-6 | 0.5 | — | — | 95.9 |
| 86 | — | D-7 | 0.5 | — | — | 92.5 |
| 87 | — | D-8 | 0.5 | — | — | 95.8 |
| 88 | — | D-9 | 0.5 | — | 92.4 | |
| 89 | — | D-10 | 0.5 | — | — | 92.8 |
| 90 | — | D-11 | 0.5 | — | — | 94.1 |
| 91 | — | D-12 | 0.5 | — | — | 94.0 |
| 92 | — | D-13 | 0.5 | — | — | 94.3 |
| 93 | — | D-14 | 0.5 | — | 92.1 | |
| 94 | — | D-15 | 0.5 | — | 95.9 | |
| 95 | — | D-8 | — | 0.5 | — | 94.6 |
| 96 | — | D-9 | 0.5 | — | — | 97.4 |
| | — | D-10 | — | 0.5 | — | |
| — | 13 | — | — | — | — | 78.8 |
| — | 14 | D′-1 | 0.5 | — | — | 83.9 |
| — | 15 | D′-2 | 0.5 | — | — | 84.5 |
| 97 | — | D-1 | — | — | 0.5 | 95.0 |
| 98 | — | D-2 | — | — | 0.5 | 92.6 |
| 99 | — | D-3 | — | — | 0.5 | 94.6 |
| 100 | — | D-4 | — | — | 0.5 | 94.0 |
| 101 | — | D-5 | — | — | 0.5 | 92.9 |
| 102 | — | D-6 | — | — | 0.5 | 93.2 |
| 103 | — | D-7 | — | — | 0.5 | 93.6 |
| 104 | — | D-8 | — | — | 0.5 | 92.9 |
| 105 | — | D-9 | — | — | 0.5 | 95.3 |
| 106 | — | D-10 | — | — | 0.5 | 93.0 |
| 107 | — | D-11 | — | — | 0.5 | 94.3 |
| 108 | — | D-12 | — | — | 0.5 | 93.5 |
| 109 | — | D-13 | — | — | 0.5 | 94.5 |
| 110 | — | D-14 | — | — | 0.5 | 94.7 |
| 111 | — | D-15 | — | — | 0.5 | 95.4 |
| — | 16 | — | — | — | — | 79.0 |
| — | 17 | D′-1 | — | — | 0.5 | 82.4 |
| — | 18 | D′-2 | — | — | 0.5 | 85.1 |

[Fabrication of Positive Electrode and Negative Electrode for Electrical Double-Layer Capacitors]

85.0 parts of activated carbon powder and (D) in a number of parts shown in Table 4 were sufficiently mixed together in a mortar, then 70.0 parts of acetone were added, and the above materials were further sufficiently mixed in the mortar to obtain a slurry. The obtained slurry was dried under reduced pressure (1.3 kPa), and then mixed together with 7.5 parts of carbon black and 7.5 parts of polytetrafluoroethylene (PTFE) powder. The obtained mixture was kneaded in the mortar for about 5 min, and rolled by a roll press to obtain an activated carbon sheet. The thickness of the activated carbon sheet was 400 μm. This activated carbon sheet was punched into a disk shape of 20 mmΦ, so as to fabricate an activated carbon electrode.

### Comparative Example 13

Except that the electrode protection film forming agent (D) was not added, the positive electrode and the negative electrode for the electrical double-layer capacitor of Comparative Example 13 were fabricated with the same method of Example 80.

### Comparative Example 14

Except that 0.5 part of methyl phenyl sulfide (D′-1) as a comparative additive was added instead of the electrode protection film forming agent (D), the positive electrode for the electrical double-layer capacitor of Comparative Example 14 was fabricated with the same method of Example 80.

### Comparative Example 15

Except that 0.5 part of diphenyl sulfide (D′-2) as a comparative additive was added instead of the electrode protection film forming agent (D), the positive electrode for the electrical double-layer capacitor of Comparative Example 15 was fabricated with the same method of Example 80.

[Fabrication of Electrical Double-Layer Capacitor]

The electrodes of each of Examples 80 to 96 and Comparative Examples 13 to 15 were arranged in a storage case including an A1-laminated polypropylene film with the respective coated surfaces facing each other, and a separator (nonwoven fabric made from polypropylene) was inserted between the electrodes, so as to fabricate a cell for a capacitor. An electrolyte solution formed by dissolving 1-ethyl-3-methylimidazolium tetrafluoroborate (EDMI.BF₄) (by Tokyo Chemical Industry Co., Ltd.) in propylene carbonate (PC) in a proportion of 12 wt % was injected in the fabricated cell and sealed.

<Evaluation of Long-Term Durability>

The fabricated electrical double-layer capacitor was connected to a charge/discharge test device (“CDT-5R2-4”, made by Power Systems Co., Ltd.), and a charge/discharge cycle test was performed in which a constant-current charging was carried out at 25 mA until a setting voltage of 3.0 V was reached and a constant-current discharging was performed at 25 mA after 7200 seconds from the start of the charging. 250 cycles were carried out at a set temperature of 60° C., and the electrostatic capacity values and electrostatic capacity retention ratios (%) of the cell at the initial stage and after 250 cycles were measured. A higher electrostatic capacity retention ratio means a better durability, so this value was used as an index of long-term durability.

Electrostatic capacity retention ratio (%)=(electrostatic capacity after 250 cycles/initial electrostatic capacity)×100

### Examples 97 to 111 and Comparative Examples 16 to 18

### Evaluation of Electrical Double-Layer Capacitors and Electrolyte Solutions

An electrical double-layer capacitor that used an electrolyte solution containing the above electrode protection film forming agent (D) or comparative electrode protection film forming agent (D′) in a number of parts shown in Table 4 was fabricated by the method described later.

Similar to the case of the electrodes, results of evaluation of long-term durability by the above-mentioned method are shown in Table 4.

[Preparation of Electrolyte Solution]

The electrode protection film forming agent (D) in a number of parts shown in Table 4 was compounded into the nonaqueous solvent (F) including 87.5 parts of PC, and EDMI.BF₄ as the electrolyte (E) was dissolved therein so that the content of EDMI.BF₄ is 12 wt %, thereby preparing an electrolyte solution of Examples 97 to 111.

### Comparative Example 16

Except that the electrode protection film forming agent (D) was not added, the electrolyte solution of Comparative Example 16 was prepared by the same method of Example 97.

### Comparative Example 17

Except that 0.5 part of methyl phenyl sulfide (D′-1) as a comparative additive was added instead of the electrode protection film forming agent (D), the electrolyte solution of Comparative Example 17 was prepared by the same method of Example 97.

### Comparative Example 18

Except that 0.5 part of diphenyl sulfide (D′-2) as a comparative additive was added instead of the electrode protection film forming agent (D), the electrolyte solution of Comparative Example 18 was prepared by the same method of Example 97.

[Fabrication of Electrode]

85.0 parts of activated carbon powder, 7.5 parts of carbon black and 7.5 parts of polytetrafluoroethylene (PTFE) powder were mixed together. The obtained mixture was kneaded in a mortar for about 5 minutes, and rolled by a roll press to obtain an activated carbon sheet. The thickness of the activated carbon sheet was 400 μm. This activated carbon sheet was punched into a disk shape of 20 mmΦ to obtain an activated carbon electrode.

[Assembly of Capacitor Cell]

A separator (nonwoven fabric made from polypropylene) was inserted between the above-mentioned positive electrode and negative electrode. The resultant was impregnated with an electrolytic solution of Examples 97 to 111 and Comparative Examples 16 to 18, placed into a storage case including an A1-laminated polypropylene film and sealed, so as to fabricate an electrical double-layer capacitor cell.

From the results of the above Examples and Comparative Examples, it is clear that the lithium secondary battery and the lithium-ion capacitor that are fabricated by using the electrode protection film forming agent of the invention are good in charge/discharge cycle performance and high-temperature storage characteristic. A reason for the improvement in charge/discharge cycle performance and high-temperature storage characteristic is that the polymerization film formed on the surface of the electrode active material suppresses decomposition of the electrolyte solution on the surface of the electrode under a high voltage condition.

From the results of the above Examples and Comparative Examples, it is clear that the electrical double-layer capacitor fabricated by using the electrode protection film forming agent of the invention has a high capacity retention ratio and is good in long-term durability. A reason for the improvement in capacity retention ratio is that the polymerization film formed on the surface of the electrode active material suppresses decomposition of the electrolyte solution on the surface of the electrode.

### INDUSTRIAL APPLICABILITY

The electrode and electrolyte solution that use the electrode protection film forming agent (D) of the invention are useful in electrochemical devices such as Li secondary batteries, Li-ion capacitors and electrical double-layer capacitors, especially in Li secondary batteries and Li-ion capacitors for electric vehicles, and electrical double-layer capacitors for wind power generation or on-vehicle use. They can also be applied to electrochemical devices other than those disclosed in this application (e.g., Ni-metal hydride batteries, Ni—Cd batteries, air batteries and alkali batteries, etc.).

1. An electrode protection film forming agent (D), containing a compound (C) having a urethane bond (a) and a polymerizable unsaturated bond (b).

2. The electrode protection film forming agent (D) of claim 1, wherein the compound (C) is represented by general formula (1):
A(-NHCO₂—X)ₙ (1)
wherein in formula (1), A is i) an n-valent hydrocarbon group having 2 to 42 carbon atoms, ii) a trivalent residue obtained by removing three isocyanate groups from a trimer of a diisocyanate (B) having 2 to 42 carbon atoms, or iii) a divalent residue obtained by removing two isocyanate groups from a urethane prepolymer having isocyanate groups at both ends, the urethane prepolymer being a reaction product of a diisocyanate (B) having 2 to 42 carbon atoms and a diol (N) having 2 to 20 carbon atoms; X is a monovalent organic group having 3 to 42 carbon atoms and having a polymerizable unsaturated bond (b); n is an integer of 1 to 6, and when n is 2 or more, a plurality of X may be the same or different from each other.

3. The electrode protection film forming agent (D) of claim 2, wherein in general formula (1), A is a divalent group and is at least one group selected from the group consisting of an aromatic hydrocarbon group having 6 to 12 carbon atoms, an aliphatic-aromatic hydrocarbon having 6 to 42 carbon atoms group, an alicyclic hydrocarbon group having 5 to 13 carbon atoms, and a polymethylene group having 2 to 8 carbon atoms.

4. The electrode protection film forming agent (D) of claim 2, wherein in general formula (1), X is a monovalent aliphatic hydrocarbon group (X1) having 3 to 42 carbon atoms that contains one to four C—C double bonds and may contain a ring other than an aromatic ring, a monovalent hydrocarbon group (X2) having 8 to 42 carbon atoms, containing one to four C—C double bonds and having an aromatic ring, or a monovalent organic group (X3) having 3 to 42 carbon atoms and containing one to four C—C double bonds of which at least one is in a group represented by formula (2), an acryloyloxyalkyl group or a methacryloyloxyalkyl group:
C═C—O (2).

5. The electrode protection film forming agent (D) of claim 4, wherein in general formula (1), X has an aromatic ring, and in the hydrocarbon group (X1), the C—C double bond and the aromatic ring are conjugated with each other.

6. An electrode, containing the electrode protection film forming agent (D) of claim 1.

7. The electrode of claim 6, comprising a protection film formed by polymerizing the electrode protection film forming agent (D).

8. An electrode for use in a lithium secondary battery, containing the electrode protection film forming agent (D) of claim 1, or comprising a protection film formed by polymerizing the electrode protection film forming agent (D).

9. An electrode for use in a lithium-ion capacitor, containing the electrode protection film forming agent (D) of claim 1, or comprising a protection film formed by polymerizing the electrode protection film forming agent (D).

10. An electrode for use in an electrical double-layer capacitor, containing the electrode protection film forming agent (D) of claim 1, or comprising a protection film formed by polymerizing the electrode protection film forming agent (D).

11. An electrolyte solution, containing the electrode protection film forming agent (D) of claim 1, an electrolyte (E) and a nonaqueous solvent (F).

12. The electrolyte solution of claim 11 for use in a lithium secondary battery.

13. The electrolyte solution of claim 11 for use in a lithium-ion capacitor.

14. The electrolyte solution of claim 11 for use in an electrical double-layer capacitor.

15. A lithium secondary battery, comprising at least one of an electrode described below and an electrolyte solution described below, wherein
the electrode contains the electrode protection film forming agent (D) of claim 1, or a protection film formed by polymerizing the electrode protection film forming agent (D), and
the electrolyte solution contains the electrode protection film forming agent (D) of claim 1, an electrolyte (E) and a nonaqueous solvent (F).

16. A lithium-ion capacitor, comprising at least one of an electrode described below and an electrolyte solution described below, wherein
the electrode contains the electrode protection film forming agent (D) of claim 1, or a protection film formed by polymerizing the electrode protection film forming agent (D), and
the electrolyte solution contains the electrode protection film forming agent (D) of claim 1, an electrolyte (E) and a nonaqueous solvent (F).

17. An electrical double-layer capacitor, comprising at least one of an electrode described below and an electrolyte solution described below, wherein
the electrode contains the electrode protection film forming agent (D) of claim 1, or a protection film formed by polymerizing the electrode protection film forming agent (D), and
the electrolyte solution contains the electrode protection film forming agent (D) of claim 1, an electrolyte (E) and a nonaqueous solvent (F).

18. A method for fabricating an electrode protection film, comprising a step of applying a voltage after the electrode protection film forming agent (D) of claim 1 is contained in at least one of an electrode and an electrolyte solution.
